SEQUENCE LISTING (10 sequences)

SEQ ID NO: 1 - DNA, 19 bp, Artificial Sequence
  Description of Artificial Sequence  Synthetic Primer
ggtttggtgaggtgtgctc

SEQ ID NO: 2 - DNA, 19 bp, Artificial Sequence
  Description of Artificial Sequence  Synthetic Primer
tgatctttcccggaactgc

SEQ ID NO: 3 - DNA, 20 bp, Artificial Sequence
  Description of Artificial Sequence  Synthetic Primer
tctgtgcctgctgctcatag

SEQ ID NO: 4 - DNA, 20 bp, Artificial Sequence
  Description of Artificial Sequence  Synthetic Primer
tggaatcctgaacccacttc

SEQ ID NO: 5 - DNA, 20 bp, Artificial Sequence
  Description of Artificial Sequence  Synthetic Primer
atgcaccgatacacactgga

SEQ ID NO: 6 - DNA, 20 bp, Artificial Sequence
  Description of Artificial Sequence  Synthetic Primer
cgccttcctcagtaccaagt

SEQ ID NO: 7 - DNA, 20 bp, Artificial Sequence
  Description of Artificial Sequence  Synthetic Primer
aacctgcagcagactccact

SEQ ID NO: 8 - DNA, 20 bp, Artificial Sequence
  Description of Artificial Sequence  Synthetic Primer
gacacgtgtggccattgtag

SEQ ID NO: 9 - DNA, 18 bp, Artificial Sequence
  Description of Artificial Sequence  Synthetic Primer
gtcttcaccaccatggag

SEQ ID NO: 10 - DNA, 18 bp, Artificial Sequence
  Description of Artificial Sequence  Synthetic Primer
ccaccctgttgctgtagc

(19) United States
(12) Patent Application Publication
(10) Pub. No.: US 2004/0013691 A1
(43) Pub. Date: Jan. 22, 2004

(54) Immunotoxin as a therapeutic agent and uses thereof

(76) Inventor: Michael G. Rosenblum, Sugarland, TX (US)

Correspondence Address: FULBRIGHT & JAWORSKI L.L.P., 600 CONGRESS AVE., SUITE 2400, AUSTIN, TX, 78701, US

(21) Appl. No.: 10/460,774

(22) Filed: Jun. 12, 2003

Related U.S. Application Data
(60) Provisional application No. 60/388,133, filed on Jun. 12, 2002.

(51) Int. Cl. A61K 39/00; A61K 39/02; C12Q 1/00; C12Q 1/68

(52) U.S. Cl. 424/234.1; 435/4; 435/6; 424/185.1

(57) ABSTRACT

The present invention further provides insight into the mechanism of action of immunotoxins in disease states such as hyperproliferative disease states. The present invention provides a novel method of treating diseases using immunotoxins and gene expression profiling to identify genes that are modulated by immunotoxin therapy.

### BACKGROUND OF THE INVENTION

The present application claims priority to co-pending U.S. Provisional Patent Application Serial No: 60/388,133 filed on Jun. 12, 2002. The entire text of the above-referenced disclosure is specifically incorporated herein by reference without disclaimer.

1. Field of the Invention

The present invention relates generally to the fields of molecular biology, cancer biology and therapy, and toxicology. More specifically, the invention relates to gene profiling, the identification of genes involved in hyperproliferative diseases such as cancers or hyperplasias, and any diseases associated with the treatment of hyperproliferative diseases.

2. Description of Related Art

Current dogma indicates that immnotoxins kill cells by inhibition of protein synthesis. Studies have demonstrated immunotoxin conjugates or fusion proteins to be cytotoxic to cells (Atkinson et al., 2001; Bolognesi et al., 2000; Rosenblum et al., 1999; Pagliaro et al., 1998 and Kaneta et al., 1998). However, further details as to the mechanism of action of immunotoxins such as gelonin, still remains to be elucidated.

Bacterial and plant toxins, such as diphtheria toxin (DT), Pseudomonas aeruginosa toxin A, abrin, ricin, mistletoe, modeoccin, and Shigella toxin, are potent cytocidal agents due to their ability to disrupt a critical cellular function. For instance, DT and ricin inhibit cellular protein synthesis by inactivation of elongation factor-2 and inactivation of ribosomal 60s subunits, respectively (Jelajaszewicz and Wadstrom, 1978). These toxins are extremely potent because they are enzymes and act catalytically rather than stoichiometrically. The molecules of these toxins are composed of an enzymatically active polypeptide chain or fragment, commonly called “A” chain or fragment, linked to one or more polypeptide chains or fragments, commonly called “B” chains or fragments, that bind the molecule to the cell surface and enable the A chain to reach its site of action, e.g., the cytosol, and carry out its disruptive function. Access to the cytosol is referred to as “internalization”, “intoxication”, or “translocation”. These protein toxins belong to a class bearing two chains referred to as A and B chains. The B chain has the ability to bind to almost all cells whereas the cytotoxic activity is exhibited by the A chain. It is believed that the A chain must be timely liberated from the B chain, frequently by reduction of a disulfide bond, in order to make the A chain functional. These natural toxins are generally not selective for a given cell or tissue type because their B chains recognize and bind to receptors that are present on a variety of cells.

The availability of a toxin molecule which is not cytotoxic to a variety of cells when administered alone has been limited. Utilizing certain naturally occurring single chain toxin molecules which do not themselves bind to cell surface receptors and, therefore, are not normally internalized by cells, has provided toxic molecules which are relatively non-toxic to most, if not all, cells when administered alone. Such naturally occurring single chain toxins known to date, include, but are not limited to, pokeweed antiviral protein (Ramakrishnan and Houston, 1984); saponin (Thorpe, et al., 1985); and gelonin (Stirpe et al., 1980). These proteins are nontoxic to cells in the free form, but can inhibit protein synthesis once they gain entry into the cell. However, the availability of these single chain toxins in substantially pure form is limited due to the fact that they must be purified from plant sources in which they occur in relatively low amounts and the reproducibility of the concentration of the toxin in the plants is dependent upon plant growth conditions and plant harvest conditions. Other such toxins include ricin, abrin, pokeweed antiviral protein, gelonin, pseudomonas exotoxin A diptheria toxin, and alpha-sarcin.

### SUMMARY OF THE INVENTION

The present invention overcomes the deficiencies in the art by providing a novel approach to treating a disease by identifying genes that are involved in a disease state, and therapeutic agents thereof, using immunotoxin therapy and assessing gene expression. Thus, the present invention provides a method of identifying one or more genes or gene products that responds to immunotoxin therapy comprising administering an immunotoxin to a cell and determining one or more genes or gene products whose expression is upregulated or downregulated in response to the immunotoxin therapy

The present invention further provides a method of identifying one or more genes or gene products comprising assessing the expression of the one or more genes or gene products both before and after administration of the immunotoxin to the cell.

The present invention also provides a method of identifying genes that are upregulated or downregulated in response to immunotoxin therapy, further characterized as comprising: (a) administering the immunotoxin to a patient or a cell; and (b) identifying the one or more immunotoxin regulated genes or gene products that are upregulated or downregulated in response to the immunotoxin administration.

The present invention also provides a method of identifying a therapeutic agent or treatment regimen that will complement immunotoxin therapy comprising the steps of: (a) identifying one or more regulated genes or gene products that are upregulated or downregulated in response to immunotoxin therapy in a patient undergoing immunotoxin therapy; (b) identifying one or more second agents or therapies that will promote a further upregulation or downregulation of one or more of the immunotoxin regulated genes. The method further comprises administering the second agent or therapy to a patient.

The invention further provides a method of treating a patient with a hyperproliferative disease or condition comprising the steps of: (a) administering to the patient an amount of an immunotoxin that is effective to treat a disease that is amenable to such immunotoxin therapy; and (b) administering to the patient an effective amount of a therapeutic agent or treatment regimen that is selected from the immunotoxin based changes in gene expression. The therapeutic agent or treatment regimen may be selected through the practice of the method of identifying one or more genes or gene products that responds to immunotoxin therapy comprising administering an immunotoxin to a cell and determining one or more genes or gene products whose expression is upregulated or downregulated in response to the immunotoxin therapy.

A gene or gene product identified as being downregulated by immunotoxin therapy may be selected from the group consisting of the genes listed in Table II but is not limited to such. One example of a gene or gene product identified as being downregulated by immunotoxin therapy in a study of the present invention is topoisomerase II which is involved in catalyzing the relaxation of supercoiled DNA by transient cleavage and religation of both strands of the DNA helix. Thus, in accordance with the methods of the present invention inhibitors of topoisomerase II such as etoposide and doxorubicin, may be identified and employed as therapeutic agents that further promote the downregulation of topoisomerase gene expression and activity and cellular products thereof. These therapeutic agents may then be administered to a patient in combination with immunotoxin therapy to treat a disease such as a hyperproliferative disease by downregulating topoisomerase II gene expression and activity and cellular thereof.

Another example of a gene or gene product identified as being downregulated by immunotoxin therapy in a study of the present invention, is spermine synthase. Spermine belongs to the group of polyamines which are essential for cell proliferation, differentiation and transformation, and is often found to be abundant in human tumors. Thus, in accordance with the methods of the present invention, inhibitors of spermine synthase such as the polyamine inhibitors N-(3-aminopropyl)cyclohexylamine (APCHA), N-cyclohexyl-1,3-diaminopropane (C-DAP), N-(n-butyl)-1,3-diaminopropane, S-adenosyl-1,12-diamino-3-thio-9-azadodecane (AdoDatad), difluoromethylomithine (DFMO), methyl glyoxal his guanylhydrazone (MGBG), and methylglyoxal-bis(cyclopentylamidinohydrazone) MGBCP may be identified and employed as therapeutic agents to further promote the downregulation of spermine synthase expression and activity, and cellular products thereof. These therapeutic agents, in accordance with the present invention, may be administered to a patient in combination with immunotoxin therapy to treat a disease such as a hyperproliferative disease, by downregulating spermine synthase expression and activity.

A gene or gene product identified as being upregulated by immunotoxin therapy may be selected from the group consisting of the genes listed in Table II but is not limited to such. One example of a gene or gene product identified as being upregulated by immunotoxin therapy in a study of the present invention is E-selectin. E-selectin (endothelial leukocyte adhesion molecule-1) is expressed by cytokine-stimulated endothelial cells. These proteins are part of the selectin family of cell adhesion molecules and are thought to be responsible for the accumulation of blood leukocytes at sites of inflammation by mediating the adhesion of cells to the vascular lining. Adhesion molecules participate in the interaction between leukocytes and the endothelium and appear to be involved in the pathogenesis of atherosclerosis. Thus, in accordance with the methods of the present invention, inducers of E-selectin such as TNF, lipopolysaccharide (LPS), lymphotoxin, or IL-1 may be identified and employed as therapeutic agents to further promote the upregulation of E-selectin expression and activity, and cellular products thereof. These therapeutic agents, in accordance with the present invention, may be administered to a patient in combination with immunotoxin therapy to treat a disease such as a hyperproliferative disease, by upregulating E-selectin expression and activity.

Yet another example of a gene or gene product identified as being upregulated by immunotoxin therapy in a study of the present invention is cytokine A2 also known as SCYA2 or MCP-1. This gene is one of several cytokine genes clustered on the q-arm of chromosome 17. Cytokines are a family of secreted proteins involved in immunoregulatory and inflammatory processes. This cytokine displays chemotactic activity for monocytes and basophils but not for neutrophils or eosinophils. It has been implicated in the pathogenesis of diseases characterized by monocytic infiltrates, like psoriasis, rheumatoid arthritis and atherosclerosis. Thus, in accordance with the methods of the present invention, inducers of cytokine A2 such as heme, lysophosphatidylcholine, interferon-gamma, IL-17, TNF, and IL-4 may be identified and employed as therapeutic agents to further promote the upregulation of cytokine A2 expression and activity, and cellular products thereof. These therapeutic agents, in accordance with the present invention, may be administered to a patient in combination with immunotoxin therapy to treat a disease such as a hyperproliferative disease, by upregulating cytokine A2 expression and activity.

Yet another example of a gene or gene product identified as being upregulated by immunotoxin therapy in a study of the present invention is TNF-α induced protein 3. This gene was identified as a gene whose expression is rapidly induced by the tumor necrosis factor (TNF). The protein encoded by this gene is a zinc finger protein, and has been shown to inhibit NFκB activation as well as TNF-mediated apoptosis. Knockout studies of a similar gene in mice suggested that this gene is critical for limiting inflammation by terminating TNF-induced NFκB responses. Thus, in accordance with the methods of the present invention, inducers of TNF-α induced protein 3 such as TRAIL, Fas, CD40, phorbol myristate acetate (PMA), UV, EBV, IL-1, or LPS may be identified and employed as therapeutic agents to further promote the upregulation of TNF-α induced protein 3 expression and activity, and cellular products thereof. These therapeutic agents, in accordance with the present invention, may be administered to a patient in combination with immunotoxin therapy to treat a disease such as a hyperproliferative disease, by upregulating TNF-α induced protein 3 expression and activity.

In still yet another example, a gene or gene product identified as being upregulated by immunotoxin therapy in a study of the present invention is NFκB inhibitor alpha also known as IκBA or NFκBIA. NFκB1 binds to REL, RELA or RELB to form the NFκB complex. This complex is inhibited by IKB proteins (e.g. NFκBIA), which inactivates NFκB by cytoplasmic trapping. Activated NFκB complex translocates into the nucleus and binds DNA at κB-binding motifs, activating gene expression. Previous studies have shown that the inappropriate activation of NFκB has been linked to inflammatory events associated with autoimmune arthritis, asthma, septic shock, lung fibrosis, atherosclerosis, and AIDS. In contrast, complete and persistent inhibition of NFκB has been linked directly to apoptosis, inappropriate immune cell development, and delayed cell growth. Thus, in accordance with the methods of the present invention, inducers of NFκB inhibitor alpha such as REIA, V-REL or deoxycholate(DOC) may be identified and employed as therapeutic agents to further promote the upregulation of NFκB inhibitor alpha expression and activity, and cellular products thereof. These therapeutic agents, in accordance with the present invention, may be administered to a patient in combination with immunotoxin therapy to treat a disease such as a hyperproliferative disease, by upregulating NFκB inhibitor alpha expression and activity.

Thus, the therapeutic agents may be administered to a patient in combination with immunotoxin therapy to treat a disease by downregulating a gene selected from the group consisting of the genes listed in Table II, or by upregulating a gene selected from the group consisting of the genes listed in Table III.

The therapeutic agent of the present invention may be an immunotoxin, fusion protein or immunoconjugate thereof, a protein or a nucleic acid expression construct such as an antisense construct; or a small molecule or organo-pharmaceutical. The therapeutic agent(s) of the present invention may be a DNA damaging agent, an alkylating agent, or an antitumor agent, but is not limited to such. The invention may also employ treatment regimens such as radiotherapy, immunotherapy, hormonal therapy or gene therapy.

Administration of immunotoxin therapy and/or a therapeutic agent may be by systemic intravenous injection, regional administration via blood or lymph supply, or directly to an affected site.

In the present invention, the cell may be a cell in a diseased state. Such as cell may be a hyperproliferative cell such as a cancer cell or an atherosclerosis cell, but is not limited to such. The cell may be located in a mammal such as a human, or in a cell culture.

The present invention also provides a method of treatment of any disease for which immunotoxin therapy can be utilized such as hyperproliferative diseases or other related disorders. One such hyperproliferative disease for which immunotoxin therapy may be used is a cancer. Cancers that may be treated include, but are not limited to, cancers of the bladder, blood, bone, bone marrow, brain, breast, colon, esophagus, gastrointestine, gums, head, kidney, liver, lung, nasopharynx, neck, ovary, prostate, skin, stomach, testis, tongue, or uterus. In some embodiments of the present invention, the hyperproliferative disease or condition being treated using immunotoxin therapy is atherosclerosis.

The use of the word “a” or “an” when used in conjunction with the term “comprising” in the claims and/or the specification may mean “one,” but it is also consistent with the meaning of “one or more,” “at least one,” and “one or more than one.”

Any of the methods and compositions disclosed herein may be applied to any other methods and compositions described herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

### I. THE PRESENT INVENTION

The present invention provides novel methods for treating diseases using immunotoxin therapy and gene expression profiling to identify genes involved in a diseases state. The present invention further provides a novel approach to identifying therapuetic agents for the treatment of diseases such as, but not limited to, hyperproliferative diseases. By identification of genes that are upregulated or downregulated in response to immunotoxin therapy, the present invention further identifies therapeutic agents that further promote the inhibition or induction of the immunotoxin regulated genes. The present invention further provides a method of treating patients using immunotoxin therapy in combination with a therapuetic agent(s).

### II. IMMUNOTOXINS

Gelonin is a single chain polypeptide isolated from seeds of a plant, Gelonium multiforum, having a molecular weight of approximately 28,000-30,000 kd. Gelonin is a basic glycoprotein with an approximate isoelectric point of 8.15 and contains mannose and glucosamine residues (Falasca, et al., 1982). Gelonin is a type I ribosome inactivating protein and an extremely potent inhibitor of protein synthesis, similar to the other known toxin ricin. Type I toxins possess the catalytic A chain necessary for protein synthesis inhibition but lack the B-chain that is characteristic of the type II toxins such as ricin. Gelonin is a 258 amino acid containing lysine residues and shares homology with that of trichosanthin and ricin A chain (Rosenblum et al., 1995).

In contrast to other plant and bacterial toxins, this protein is not toxic to cells by itself, but when delivered to cells through a carrier, it damages the 60s ribosomal subunit. In vivo and in vitro biological data suggest that gelonin is equivalent or superior to other plant toxins. Studies comparing gelonin conjugates in vitro and in vivo with other A chain conjugates indicated that gelonin had similar potency, better selectivity, better tumor localization, and more significant therapeutic effects (Sivan, et al., 1987). However, the availability of a reproducible, readily accessible supply of gelonin from natural sources is limited. In addition, the purification of gelonin from plant sources is difficult and the yield is very low.

Gelonin by itself has been shown to be abortifacient in mice and enhances antibody dependent cell cytotoxicity (Yeung, et al (1988). Several investigators have utilized gelonin as a cytotoxic agent chemically attached to monoclonal antibodies or to peptide hormone cellular targeting ligands (Atkinson et al., 2001; Bolognesi et al., 2000; Rosenblum et al., 1999; Pagliaro et al., 1998 and Kaneta et al., 1998).

Recombinant gelonin may also be produced for use in the preseent invention as described in U.S. Pat. No. RE37,462, and Rosenblum et al., 1995, each incorporated herein by reference. In some instances, recombinant gelonin may be produced using the cDNA of gelonin. Recombinants of the present invention may be produced by introducing mutations into the molecule. Recombinant gelonin can be produced by site directed mutagenesis to have greater toxic activity than the native molecule; to be more effectively internalized once bound to the cell surface by a carrier such as a monoclonal antibody or a targeting ligand to resist lysosomal degradation and thus be more stable and longer acting as a toxic moiety. Recombinant gelonin may also be produced by engineering fusion products to contain other functional modalities to kill cells such as an enzymes, cytokines (TNF or IFN), or a second toxin, such as diptheria toxin, thus creating a “supertoxin” or a toxin with multifunctional actions. Fusion proteins can be engineered with gelonin to carry drugs such as chemotherapeutic agents. Gelonin peptides may have application as abortofacient agents, immunosuppressive agents, anticancer agents and as antiviral agents.

A. Immunotoxin Antibodies

The toxins of the present invention are particularly suited for use as components of cytotoxic therapeutic agents. To form cytotoxic agents, immunotoxin toxins of the present invention may be conjugated to monoclonal antibodies, including chimeric and CDR-grafted antibodies, and antibody domains/fragments (e.g., Fab, Fab′, F(ab′).sub.2, single chain antibodies, and Fv or single variable domains). An immunotoxin may also consist of a fusion protein rather than an immunoconjugate. Immunoconjugates including toxins may be described as immunotoxins.

Immunotoxin toxins conjugated to monoclonal antibodies genetically engineered to include free cysteine residues are also within the scope of the present invention. Examples of Fab′ and F(ab′).sub.2 fragments useful in the present invention are described in WO 89/00999, which is incorporated by reference herein. Alternatively, the immunotoxin toxins may be conjugated or fused to humanized or human engineered antibodies. Such humanized antibodies may be constructed from mouse antibody variable domains.

1. Antibody Regions

Regions from the various members of the immunoglobulin family are encompassed by the present invention. Both variable regions from specific antibodies are covered within the present invention, including complementarity determining regions (CDRs), as are antibody neutralizing regions, including those that bind effector molecules such as Fe regions. Antigen specific-encoding regions from antibodies, such as variable regions from IgGs, IgMs, or IgAs, can be employed with the pIgR-binding domain in combination with an antibody neutralization region or with one of the therapeutic compounds described herein.

In particular embodiments, the present invention may comprise a single-chain antibody. Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule.

Single-chain antibody variable fragments (scFvs) in which the C-terminus of one variable domain is tethered to the N-terminus of the other via a 15 to 25 amino acid peptide or linker, have been developed without significantly disrupting antigen binding or specificity of the binding (Bedzyk et al., 1990; Chaudhary et al., 1990). These Fvs lack the constant regions (Fc) present in the heavy and light chains of the native antibody. Immunotoxins employing single-chain antibodies are described in U.S. Pat. No. 6,099,842, specifically incorporated by reference.

Antibodies to a wide variety of molecules are contemplated, such as oncogenes, tumor-associated antigens, cytokines, growth factors, hormones, enzymes, transcription factors or receptors. Also contemplated are secreted antibodies targeted against serum, angiogenic factors (VEGF/NVPF; βFGF; αFGF; and others), coagulation factors, and endothelial antigens necessary for angiogenesis (i.e., V3 integrin). Also contemplated are growth factors such as transforming growth factor, fibroblast growth factor, and platelet derived growth factor (PDGF) and PDGF family members.

The antibodies employed in the present invention as part of an immunotoxin may be targeted to any antigen. The antigen may be specific to an organism, to a cell type, to a disease or condition. Exemplary antigens include cell surface cellular proteins, for example tumor-associated antigens, viral proteins, microbial proteins, post-translational modifications or carbohydrates, and receptors. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

### III. IDENTIFYING IMMUNOTOXIN REGULATED GENES

The present invention, in various embodiments, involves identifying immunotoxin regulated genes. As is known to one of ordinary skill in the art, there are a wide variety of methods for assessing gene expression, most of which are reliant on hybrdization analysis. In specific embodiments, template-based amplification methods are used to generate (quantitatively) detectable amounts of gene products, which are assessed in various manners.

One method of identfying immunotoxin regulated genes may employ DNA or cDNA arrays technology which provides a means of rapidly screening a large number of DNA samples for their ability to hybridize to a variety of single stranded DNA probes immobilized on a solid substrate. Specifically contemplated are cDNA microarray technologies. Micro-array technology, a hybridization-based process that allows simultaneous quantitation of many nucleic acid species, has been described (Schena et al., 1995 and 1996; DeRisi et al., 1996). This technique combines robotic spotting of small amounts of individual, pure nucleic acid species on a glass surface, hybridization to this array with multiple fluorescently labeled nucleic acids, and detection and quantitation of the resulting fluor tagged hybrids with a scanning confocal microscope. When used to detect transcripts, a particular RNA transcript (an mRNA) is copied into DNA (a cDNA) and this copied form of the transcript is immobilized on a glass surface. The entire complement of transcript mRNAs present in a particular cell type is extracted from cells and then a fluor-tagged cDNA representation of the extracted mRNAs is made in vitro by an enzymatic reaction termed reverse-transcription. Fluor-tagged representations of mRNA from several cell types, each tagged with a fluor emitting a different color light, are hybridized to the array of cDNAs and then fluorescence at the site of each immobilized cDNA is quantitated. The various characteristics of this analytic scheme make it particularly useful for directly comparing the abundance of mRNAs present in two cell types. Visual inspection of such a comparison is sufficient to find genes where there is a very large differential rate of expression.

### IV. ANTISENSE CONSTRUCTS

The present invention may further employ antisense constructs directed to downregulating a particular gene. The term “antisense nucleic acid” is intended to refer to the oligonucleotides complementary to the base sequences of DNA and RNA. Antisense oligonucleotides, when introduced into a target cell, specifically bind to their target nucleic acid and interfere with transcription, RNA processing, transport and/or translation. Targeting double-stranded (ds) DNA with oligonucleotide leads to triple-helix formation; targeting RNA will lead to double-helix formation.

Antisense constructs may be designed to bind to the promoter and other control regions, exons, introns or even exon-intron boundaries of a gene, as is known those of skill in the art. Antisense RNA constructs, or DNA encoding such antisense RNAs, may be employed to inhibit gene transcription or translation or both within a host cell, either in vitro or in vivo, such as within a host animal, including a human subject. Nucleic acid sequences comprising “complementary nucleotides” are those which are capable of base-pairing according to the standard Watson-Crick complementary rules. That is, that the larger purines will base pair with the smaller pyrimidines to form only combinations of guanine paired with cytosine (G:C) and adenine paired with either thymine (A:T), in the case of DNA, or adenine paired with uracil (A:U) in the case of RNA.

As used herein, the terms “complementary” or “antisense sequences” mean nucleic acid sequences that are substantially complementary over their entire length and have very few base mismatches. For example, nucleic acid sequences of fifteen bases in length may be termed complementary when they have a complementary nucleotide at thirteen or fourteen positions with only single or double mismatches. Naturally, nucleic acid sequences which are “completely complementary” will be nucleic acid sequences which are entirely complementary throughout their entire length and have no base mismatches.

While all or part of the gene sequence may be employed in the context of antisense construction, statistically, any sequence 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that oligonucleotides of 8, 9, 10, 11, 12, 13, 14, 15, 16, 17, 18, 19, 20 or more base pairs will be used. One can readily determine whether a given antisense nucleic acid is effective at targeting of the corresponding host cell gene simply by testing the constructs in vitro to determine whether the endogenous gene's function is affected or whether the expression of related genes having complementary sequences is affected.

In certain embodiments, one may wish to employ antisense constructs which include other elements, for example, those which include C-5 propyne pyrimidines. Oligonucleotides which contain C-5 propyne analogues of uridine and cytidine have been shown to bind RNA with high affinity and to be potent antisense inhibitors of gene expression (Wagner et al., 1993).

As an alternative to targeted antisense delivery, targeted ribozymes may be used. The term “ribozyme” refers to an RNA-based enzyme capable of targeting and cleaving particular base sequences in oncogene DNA and RNA. Ribozymes either can be targeted directly to cells, in the form of RNA oligo-nucleotides incorporating ribozyme sequences, or introduced into the cell as an expression construct encoding the desired ribozymal RNA. Ribozymes may be used and applied in much the same way as described for antisense nucleic acids.

### V. COMBINATION THERAPIES

It is contemplated that a wide variety of conditions or diseases may be treated, using compositions and methods of the present invention. Hyperproliferative diseases or disorders such as cancer are specifically contemplated. Cancers that can be treated with the present invention include, but are not limited to, hematological malignancies including: blood cancer, myeloid leukemia, monocytic leukemia, myelocytic leukemia, promyelocytic leukemia, myeloblastic leukemia, lymphocytic leukemia, acute myelogenous leukemic, chronic myelogenous leukemic, lymphoblastic leukemia, hairy cell leukemia, and acute lymphocytic leukemia. Solid cell tumors and cancers that can be treated include those such as tumors of the brain (glioblastomas, medulloblastoma, astrocytoma, oligodendroglioma, ependymomas), lung, liver, spleen, kidney, lymph node, small intestine, pancreas, colon, stomach, breast, endometrium, prostate, testicle, ovary, skin, head and neck, esophagus, bladder. Other cancers and tumors such as bronchogenic oat-cell carcinoma, non-small cell lung carcinoma, retinoblastoma, neuroblastoma, mycosis fungoides, Wilms' tumor, Hodgkin's disease, osteogenic sarcoma, soft tissue sarcoma, Ewing's sarcoma, rhabdomyosarcoma may also be treated using compositions and methods of the present invention. Furthermore, the cancer may be a precancer, a metastatic and/or a non-metastatic cancer.

It may be desirable to combine the immunotoxin therapy of the present invention with an agent effective in the treatment of a disease such as a hyperproliferative diseases or disorders. In some embodiments, it is contemplated that a conventional therapy or agent, including but not limited to, a pharmacological therapeutic agent, a surgical therapeutic agent (e.g., a surgical procedure) or a combination thereof, may be combined with treatment directed to a gene target. In certain embodiments, a therapeutic method of the present invention may comprise increasing or decreasing the expression of a gene in combination with more that one additional therapeutic agents.

This process may involve contacting the cell(s) with an agent(s) and the immunotoxin at the same time or within a period of time wherein separate administration of the immunotoxin and an agent to a cell, tissue or organism produces a desired therapeutic benefit. The terms “contacted” and “exposed,” when applied to a cell, tissue or organism, are used herein to describe the process by which the immunotoxin and/or therapeutic agent are delivered to a target cell, tissue or organism or are placed in direct juxtaposition with the target cell, tissue or organism. The cell, tissue or organism may be contacted (e.g., by adminstration) with a single composition or pharmacological formulation that includes both a immunotoxin and one or more agents, or by contacting the cell with two or more distinct compositions or formulations, wherein one composition includes a immunotoxin and the other includes one or more agents.

The immunotoxin may precede, be co-current with and/or follow the other agent(s) by intervals ranging from minutes to weeks. In embodiments where the immunotoxin and other agent(s) are applied separately to a cell, tissue or organism, one would generally ensure that a significant period of time did not expire between the time of each delivery, such that the immunotoxin and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the immunotoxin. In other aspects, one or more agents may be administered within of from substantially simultaneously, about 1 minute, about 5 minutes, about 10 minutes, about 20 minutes, about 30 minutes, about 45 minutes, about 60 minutes, about 2 hours, about 3 hours, about 4 hours, about 5 hours, about 6 hours, about 7 hours, about 8 hours, about 12 hours, about 18 hours, about 24 hours, about 36 hours, about 48 hours, about 1 day, about 2 days, about 3 days, about 4 days, about 5 days, about 6 days, about 7 days, about 14 days, about 21 days, about 4 weeks, about 5 weeks, about 6 weeks, about 7 weeks, about 8 weeks, about 3 months, about 4 months, about 5 months, about 6 months, about 7 months, about 8 months, about 9 months, about 10 months, about 11 months, or about 12 months, and any range derivable therein, prior to and/or after administering the immunotoxin.

It also is conceivable that more than one administration of either the other chemotherapeutic and the immunotoxin will be required to achieve complete cancer cure. It is also contemplated that more than one administration of either the second therapeutic agent or the immunotoxin, or any agents of the present invention will be administered in an effective amount as a therapeutic modality. An “effective amount as used herein is defined as an amount of the agent that will induce or inhibit a particular gene(s) and further decrease, inhibit or otherwise abrogate the disease.

Various combinations of therapies may be employed, in which a composition comprising an immunotoxin is “A” and the secondary agent is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Other combinations are also contemplated. The exact dosages and regimens of each agent can be suitable altered by those of ordinary skill in the art. In particular embodiments, the immunotoxin of the present invention may be administered before, after, or at the same time as the secondary agent or other therapy.

A. Therapeutic Agents

Therapeutic agents and methods of administration, dosages, etc., are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

B. Therapeutic Agents of Hyperproliferative Diseases

Hyperproliferative diseases include cancer, for which there is a wide variety of treatment regimens such as anti-cancer agents or surgery. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

Anti-cancer agents include biological agents (biotherapy), chemotherapy agents, and radiotherapy agents. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that therapy with immunotoxin could be used similarly in conjunction with chemotherapeutic, radiotherapeutic, immunotherapeutic or other biological intervention, in addition to other pro-apoptotic or cell cycle regulating agents.

1. Chemotherapeutic Agents

A wide variety of chemotherapeutic agents may be used in combination with the immunotoxin of the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell. An agent may be characterized based on its ability to directly cross-link DNA, to intercalate into DNA, or to induce chromosomal and mitotic aberrations by affecting nucleic acid synthesis. An agent may also be characterized by its ability to induce or inhibit gene expression.

Most chemotherapeutic agents fall into the categories of alkylating agents, antimetabolites, antitumor antibiotics, corticosteroid hormones, mitotic inhibitors, and nitrosoureas, but are not limited to these categories. It is contemplated that immunotoxin can be used in combination with one or more of these agents according to the present invention.

a. Alkylating Agents

Alkylating agents are drugs that directly interact with genomic DNA to prevent the cancer cell from proliferating and may be used in combination with the present invention. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of-the breast, lung, and ovary. They include but are not limited to: busulfan (myleran), chlorambucil, cisplatin, cyclophosphamide (cytoxan), dacarbazine, ifosfamide, mechlorethamine (mustargen), and melphalan (also known as alkeran, L-phenylalanine mustard, phenylalanine mustard, L-PAM, or L-sacrolysin). Immunotoxin can be used to treat cancer in combination with any one or more of these alkylating agents, or analogs or derivatives thereof.

b. Antimetabolites

Antimetabolites disrupt DNA and RNA synthesis and may also be used in combination with the present invention. Unlike alkylating agents, they specifically influence the cell cycle during S phase. They have been used to combat chronic leukemias in addition to tumors of breast, ovary and the gastrointestinal tract. Antimetabolites include but are not limited to: 5-fluorouracil (5-FU), cytarabine (Ara-C), fludarabine, gemcitabine, and methotrexate, or analogs or derivatives thereof.

c. Antitumor Antibiotics

Antitumor antibiotics have both antimicrobial and cytotoxic activity and may also be used in combination with the present invention. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include but are not limited to: bleomycin, actinomycin D (dactinomycin), daunorubicin, doxorubicin (Adriamycin), mitomycin (also known as mutamycin and/or mitomycin-C), plicomycin, and idarubicin, anthracyline and anthracyclinones or analogs or derivatives thereof.

d. Corticosteroid Hormones

Corticosteroid hormones are useful in treating some types of cancer (lymphoma, leukemias, and multiple myeloma) and may also be used in combination with the present invention. Though these hormones have been used in the treatment of many non-cancer conditions, they are considered chemotherapy drugs when they are implemented to kill or slow the growth of cancer cells. Corticosteroid hormones include but are not limited to: prednisone and dexamethasone or analogs or derivatives thereof.

e. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, vinblastine, vincristine, and vinorelbine, or analogs or derivatives thereof. These inhibitors may also be used in combination with the present invention as a therapuetic modality.

f. Nitrosureas

Nitrosureas, like alkylating agents, inhibit DNA repair proteins. They are used to treat non-Hodgkin's lymphomas, multiple myeloma, malignant melanoma, in addition to brain tumors. Examples include but are not limited to carmustine and lomustine, or analogs or derivatives thereof.

g. Miscellaneous Agents

Other chemotherapy agents contemplated that may be employed with the present invention for use in combination therapies of cancer include but are not limited to: amsacrine, L-asparaginase, retinoids such as tretinoin, and tumor necrosis factor (TNF), or analogs or derivatives thereof.

2. Adjunct Therapies

Other agents or therapies may also be used in combination with the present invention. These include by are not limited to radiotherapy, immunotherapy, gene therapy, and hormonal therapy.

a. Radiotherapy

Other factors that cause DNA damage and have been used extensively include γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

b. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

The general approach for combined therapy is discussed below. In one aspect the immunotherapy can be used to target a tumor cell. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. Alternate immune stimulating molecules also exist including cytokines such as: interleukin 1 (IL-1), IL-2, IL-3, IL-4, IL-5, IL-6, IL-7, IL-8, IL-9, IL-10, IL-11, IL-12, IL-13, IL-14, IL-15, β-interferon, α-interferon, γ-interferon, angiostatin, thrombospondin, endostatin, METH-1, METH-2, Flk2/Flt3 ligand, GM-CSF, G-CSF, M-CSF, and tumor necrosis factor (TNF), chemokines such as MIP-1, MCP-1, and growth factors such as FLT3 ligand. Combining immune stimulating molecules, either as proteins or using gene delivery in combination with immunotoxin based combination therapy of the present invention will enhance anti-tumor effects.

c. Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

d. Active Immunotherapy

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton and Ravindranath, 1996; Morton et al, 1992; Mitchell et al., 1990; Mitchell et al., 1993).

e. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro.

3. Gene Therapy

In particular embodiments, the secondary treatment is gene therapy in which the immunotoxin of the present invention is contemplated. A variety of proteins are encompassed within the invention, which include but is not limited to inhibitors of cellular proliferation and regulators of programmed cell death. Table 1 below lists various genes that may be targeted for gene therapy of some form in combination with the present invention.

a. Inhibitors of Cellular Proliferation

Tumor suppressors function to inhibit excessive cellular proliferation. The inactivation of these genes destroys their inhibitory activity, resulting in unregulated proliferation. Thus it is contemplated that the present invention may be combined with tumor suppressor such as p53, p16 and C-CAM.

Other genes that may be employed according to the present invention include Rb, APC, mda-7, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

b. Regulators of Programmed Cell Death

The Bcl-2 family of proteins and ICE-like proteases have been demonstrated to be important regulators and effectors of apoptosis in other systems. The Bcl-2 protein, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). Thus, it is contemplated that Bcl-2 and the Bcl-2 family of anti-apoptotic proteins (e.g., Bcl_{XL}, Bcl_{W}, Bcl_{S}, Mcl-1, Al, Bf1-1), or the Bcl-2 family of pro-apoptotic proteins (e.g., Bax, Bak, Bik, Bim, Bid, Bad, Harakiri) may be employed with the present invention.

c. Growth Factors

In other embodiments, the present invention may employ growth factors or ligands. Examples include VEGF/VPF, FGF, TGFβ, ligands that bind to a TIE, tumor-associated fibronectin isoforms, scatter factor, hepatocyte growth factor, fibroblast growth factor, platelet factor (PF4), PDGF, KIT ligand (KL), colony stimulating factors (CSFs), LIF, and TIMP.

TABLE 1

| | | | |
|---|---|---|---|
| Gene | Source | Human Disease | Function |

GROWTH FACTORS

| | | |
|---|---|---|
| HST/KS | Transfection | FGF family member |
| INT-2 | MMTV promoter | FGF family member |
| | Insertion | |
| INTI/ | MMTV promoter | Factor-like |
| WNTI | Insertion | |
| SIS | Simian sarcoma virus | PDGF B |

RECEPTOR TYROSINE KINASES

| | | | |
|---|---|---|---|
| ERBB/ | Avian erythroblastosis | Amplified, de- | EGF/TGF-/ |
| HER | virus; ALV promoter | leted squamous | Amphiregulin/ |
| | insertion; amplified | cell cancer; | Hetacellulin |
| | human tumors | glioblastoma | receptor |
| ERBB-2/ | Transfected from rat | Amplified | Regulated by NDF/ |
| NEU/ | Glioblastomas | breast, ovarian, | Heregulin and EGF- |
| HER-2 | | gastric cancers | Related factors |
| FMS | SM feline sarcoma | | CSF-1 receptor |
| | virus | | |
| KIT | HZ feline sarcoma | | MGF/Steel receptor |
| | virus | | Hematopoieis |
| TRK | Transfection from | | NGF (nerve growth |
| | human colon cancer | | Factor) receptor |
| MET | Transfection from | | Scatter factor/HGF |
| | human osteosarcoma | | Receptor |
| RET | Translocations and | Sporadic thy- | Orphan receptor Tyr |
| | point mutations | roid cancer; | Kinase |
| | | familial medul- | |
| | | lary thyroid | |
| | | cancer; multi- | |
| | | ple endocrine | |
| | | neoplasias 2A | |
| | | and 2B | |
| ROS | URII avian sarcoma | | Orphan receptor Tyr |
| | Virus | | Kinase |
| PDGF | Translocation | Chronic | TEL(ETS-like |
| receptor | | Myelomono- | transcription factor)/ |
| | | cytic Leukemia | PDGF receptor gene |
| | | | Fusion |
| TGF- | | Colon | |
| receptor | | carcinoma mis- | |
| | | match mutation | |
| | | target | |

NONRECEPTOR TYROSINE KINASES

| | | | |
|---|---|---|---|
| ABL | Abelson Mul. V | Chronic | Interact with RB, |
| | | myelogenous | RNA polymerase, |
| | | leukemia trans- | CRK, CBL |
| | | location with | |
| | | BCR | |
| FPS/FES | Avian Fujinami SV; | | |
| | GA FeSV | | |
| LCK | Mul. V (murine | | Src family; T cell |
| | leukemia virus) pro- | | signaling; interacts |
| | moter insertion | | CD4/CD8 T cells |
| SRC | Avian Rous sarcoma | | Membrane-associa- |
| | Virus | | ted Tyr kinase with |
| | | | signaling function; |
| | | | activated by |
| | | | receptor kinases |
| YES | Avian Y73 virus | | Src family; |
| | | | signaling |

SER/THR PROTEIN KINASES

| | | |
|---|---|---|
| AKT | AKT8 murine | Regulated by |
| | retrovirus | PI(3)K; regulate |
| | | 70-kd S6 k |
| MOS | Maloney murine SV | GVBD; cystostatic |
| | | factor; MAP kinase |
| | | kinase |
| PIM-1 | Promoter insertion | |
| | Mouse | |
| RAF/MIL | 3611 murine SV; MH2 | Signaling in RAS |
| | avian SV | Pathway |

MISCELLANEOUS CELL SURFACE

| | | | |
|---|---|---|---|
| APC | Tumor suppressor | Colon cancer | Interacts with |
| | | | catenins |
| DCC | Tumor suppressor | Colon cancer | CAM domains |
| E-cadherin | Candidate tumor | Breast cancer | Extracellular homo- |
| | Suppressor | | typic binding; intra- |
| | | | cellular interacts |
| | | | with catenins |
| PTC/ | Tumor suppressor and | Nevoid basal | 12 transmembrane |
| NBCCS | Drosophilia homology | cell cancer | domain; signals |
| | | syndrome | through Gli |
| | | (Gorline | homogue CI to |
| | | syndrome) | antagonize hedge- |
| | | | hog pathway |
| TAN-1 | Translocation | T-ALL | Signaling |
| Notch | | | |
| homo- | | | |
| logue | | | |

MISCELLANEOUS SIGNALING

| | | | |
|---|---|---|---|
| BCL-2 | Translocation | B-cell | Apoptosis |
| | | lymphoma | |
| CBL | Mu Cas NS-1 V | | Tyrosine- |
| | | | Phosphorylated |
| | | | RING |
| | | | finger interact Abl |
| CRK | CT1010 ASV | | Adapted SH2/SH3 |
| | | | interact Abl |
| DPC4 | Tumor suppressor | Pancreatic | TGF--related |
| | | cancer | signaling Pathway |
| MAS | Transfection and | | Possible angiotensin |
| | Tumorigenicity | | Receptor |
| NCK | | | Adaptor SH2/SH3 |

GUANINE NUCLEOTIDE EXCHANGERS AND BINDING
PROTEINS

| | | | |
|---|---|---|---|
| BCR | | Translocated | Exchanger; protein |
| | | with ABL in | Kinase |
| | | CML | |
| DBL | Transfection | | Exchanger |
| GSP | | | |
| NF-1 | Hereditary tumor | Tumor sup- | RAS GAP |
| | Suppressor | pressor neuro- | |
| | | fibromatosis | |
| OST | Transfection | | Exchanger |
| Harvey- | HaRat SV; Ki RaSV; | Point mutations | Signal cascade |
| Kirsten, | Balb-MoMuSV; | in many human | |
| N-RAS | Transfection | tumors | |
| VAV | Transfection | | S112/S113; |
| | | | exchanger |

NUCLEAR PROTEINS AND TRANSCRIPTION FACTORS

| | | | |
|---|---|---|---|
| BRCA1 | Heritable suppressor | Mammary | Localization |
| | | cancer/ovarian | unsettled |
| | | cancer | |
| BRCA2 | Heritable suppressor | Mammary | Function unknown |
| | | cancer | |
| ERBA | Avian erythroblastosis | | thyroid hormone |
| | Virus | | receptor |
| | | | (transcription) |
| ETS | Avian E26 virus | | DNA binding |
| EVII | MuLV promotor | AML | Transcription factor |
| | Insertion | | |
| FOS | FBI/FBR murine | | transcription factor |
| | osteosarcoma viruses | | with c-JUN |
| GLI | Amplified glioma | Glioma | Zinc finger; cubitus |
| | | | interruptus homo- |
| | | | logue is in hedgehog |
| | | | signaling pathway; |
| | | | inhibitory link PTC |
| | | | and hedgehog |
| HMGI/ | Translocation t(3:12) | Lipoma | Gene fusions high |
| LIM | t(12:15) | | mobility group |
| | | | HMGI-C (XT-hook) |
| | | | and transcription |
| | | | factor LIM or |
| | | | acidic domain |
| JUN | ASV-17 | | Transcription factor |
| | | | AP-1 with FOS |
| MLL/ | Translocation/fusion | Acute myeloid | Gene fusion of |
| VHRX + | ELL with MLL | leukemia | DNA-binding and |
| ELI/MEN | Trithorax-like gene | | methyl transferase |
| | | | MLL with ELI RNA |
| | | | pol II elongation |
| | | | factor |
| MYB | Avian myeloblastosis | | DNA binding |
| | Virus | | |
| MYC | Avian MC29; | Burkitt’s | DNA binding with |
| | Translocation B-cell | lymphoma | MAX partner; |
| | Lymphomas; promoter | | cyclin regulation; |
| | Insertion avian | | interact RB; regulate |
| | leukosis Virus | | apoptosis |
| N-MYC | Amplified | Neuroblastoma | |
| L-MYC | | Lung cancer | |
| REL | Avian | | NF-B family |
| | Retriculoendotheliosis | | transcription factor |
| | Virus | | |
| SKI | Avian SKV770 | | Transcription factor |
| | Retrovirus | | |
| VHL | Heritable suppressor | Von Hippel- | Negative regulator |
| | | Landau | or elongin; trans- |
| | | syndrome | criptional elongation |
| | | | complex |
| WT-1 | | Wilm's tumor | Transcription factor |

CELL CYCLE/DNA DAMAGE RESPONSE

| | | | |
|---|---|---|---|
| ATM | Hereditary disorder | Ataxia- | Protein/lipid kinase |
| | | telangiectasia | homology; DNA |
| | | | damage response |
| | | | upstream in P53 |
| | | | pathway |
| BCL-2 | Translocation | Follicular | Apoptosis |
| | | lymphoma | |
| FACC | Point mutation | Fanconi's | |
| | | anemia group | |
| | | C (predispo- | |
| | | sition leukemia | |
| MDA-7 | Fragile site 3p14.2 | Lung | Histidine triad-re- |
| | | carcinoma | lated diadenosine |
| | | | 5,3-tetraphosphate |
| | | | asymmetric |
| | | | hydrolase |
| hML1/ | | HNPCC | Mismatch repair; |
| MutL | | | MutL Homologue |
| hMSH2/ | | HNPCC | Mismatch repair; |
| MutS | | | MutS Homologue |
| hPMS1 | | HNPCC | Mismatch repair; |
| | | | MutL Homologue |
| hPMS2 | | HNPCC | Mismatch repair; |
| | | | MutL Homologue |
| INK4/ | Adjacent INK-4B at | Candidate | p16 CDK inhibitor |
| MTS1 | 9p21; CDK complexes | MTS1 suppres- | |
| | | sor and MLM | |
| | | melanoma gene | |
| INK4B/ | | Candidate | p15 CDK inhibitor |
| MTS2 | | suppressor | |
| MDM-2 | Amplified | Sarcoma | Negative regulator |
| p53 | Association with SV40 | Mutated > 50% | p53 Transcription |
| | T antigen | human tumors, | factor; checkpoint |
| | | including | control; apoptosis |
| | | hereditary | |
| | | Li-Fraumeni | |
| | | syndrome | |
| PRAD1/ | Translocation with | Parathyroid | Cyclin D |
| BCL1 | Parathyroid hormone | adenoma; | |
| | or IgG | B-CLL | |
| RB | Hereditary | Retino- | Interact cyclin/cdk; |
| | Retinoblastoma; | blastoma; | regulate E2F |
| | Association with many | osteosarcoma; | transcription factor |
| | DNA virus tumor | breast cancer; | |
| | Antigens | other sporadic | |
| | | cancers | |
| XPA | | xeroderma | Excision repair; |
| | | pigmentosum; | photo-product |
| | | skin cancer | recognition; zinc |
| | | predisposition | finger |

4. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, agents that increase the sensitivity of the hyperproliferative cells to apoptotic inducers, or other biological agents. Immunomodulatory agents include tumor necrosis factor; and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the anti-cancer abilities of the present invention by establishment of an autocrine or paracrine effect on hyperproliferative cells. Increase intercellular signaling such as by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Studies from a number of investigators have demonstrated that tumor cells that are resistant to TRAIL can be sensitized by subtoxic concentrations of drugs/cytokines and the sensitized tumor cells are significantly killed by TRAIL. (Bonavida et al., 1999; Bonavida et al., 2000; Gliniak et al., 1999; Keane et al., 1999). Furthermore, the combination of chemotherapeutics, such as CPT-11 or doxorubicin, with TRAIL also lead to enhanced anti-tumor activity and an increase in apoptosis. Some of these effects may be mediated via up-regulation of TRAIL or cognate receptors, whereas others may not.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases. Inducers of reacctive oxyens species such as rotenone may also be used in combination with the immunotoxin of the present invention.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### VII. EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute modes for its practice. However, those of skill in the art should, in light of the present disclosure, appreciate that many changes can be made in the specific embodiments which are disclosed and still obtain a like or similar result without departing from the spirit and scope of the invention.

### Example 1

Sample Preparation for cDNA Expression Microarrays.

Human melanoma A375-M cells were treated with scFvMEL/rGel at IC₅₀ concentration (10 nM) for 24 h and untreated cells were used as a control. Approximately 1×10⁷ cells were directly lysed by addition of 5 ml of TRIzol Reagent (Life Technologies, Inc., Gaithersburg, Md.). 1 ml aliquots of the lysate were added to tubes containing 200 μl chloroform. The samples were shaken and centrifuged (15 min, 10,000×g). The upper phase was removed, placed in a clean tube containing 0.5 ml isopropyl alcohol and incubated at room temp for 10 min and then centrifuged at 10,000×g for 10 min. The RNA pellet was washed with 75% ethanol and dissolved in RNase-free water. The quality of RNA was evaluated by denaturing formaldehyde/agarose gel elctrophoresis. Microarray experiment and analysis were performed by Cancer Genomics Core Lab of M. D. Anderson Cancer Center, Houston, Tex.

### Example 2

Microarray Data Treatment and Analysis

Array Description—Sample Information. The slides were CG4.1 array design, which contains 4800 spots. Each array contained 2304 genes replicated twice, 48 positive control spots-one per grid, 48 negative control spots-one per grid and 96 blank spots.

Evaluating signal-to Noise (S/N) ratio. The signal-to-ratio of the images were evaluated to determine the quality of the array in term of how many spots had sufficient signal intensity above noise. The Signal-to-noise ratio measurement provided by the quantification software (Array Vision) is defined as: spot density minus background density, divided by the standard deviation (SD) of the background density.

In this set of arrays, a S/N>2.0 was used as a criteria to evaluate how many spots gave adequate signal (difference between signal intensity and background intensity should be greater than 2 standard deviation of the local background)

| | Cy5 | Cy5 | Cy3 | Cy3 |
|---|---|---|---|---|
| Array ID | Spots S/N > 2.0 | % | Spots S/N > 2.0 | % |
| CG041176 | 2519 | 52.5 | 3793 | 79.0 |

Normalization and threshold. For each array the background-corrected spot intensities were normalized so that the 75^{th} percentile equaled 1000, as a surrogate for the median of expressed genes. After the data sets were normalized, any spot whose normalized intensity level was below a threshold of 150 had its value replaced with the threshold value. Then the background-corrected, normalized signal intensities were log-transformed (base two) for further data analysis. The log ratio is calculated as:

$${log}_{2} ( \frac{{I}_{Cy5}}{{I}_{Cy3}} )$$

i.e., as the logarithm of the ratio of gene on Cy5 channel vs. genes on Cy3 channel.

Data treatment. A statistical method, described as the “smooth t-statistic”, was applied in order to correctly determine gene expression profiles on each array. The “smooth t-statistics” can also be interpreted as “studentized log ratios”; i.e., as estimates of the log ratio of gene expression levels between samples that have been resealed to account for the observed variability. This approach estimates both the mean log intensity and the standard deviation of the spots within a channel. In practice, since the standard deviation varies as a function of the mean, these estimates were used to fit a smooth function representing the standard deviation. After pooling these estimates of standard deviation from the two channels, a t-statistic was computed for each gene to distinguish it from the estimate that one would get by using the raw estimates of standard deviation on an independent, gene-by-gene basis. The set of all computed smooth t-statistics from a microarray are named as t-scores.

Determining “differentially expressed genes”. The first step is to flag the poorly reproducible genes on a given array (based on replicate pairs spots on an array). The criterion used to flag poorly reproducible genes is as follows: if the difference in log intensities of the replicated genes exceeds 4 times the smooth estimate of variability, then the replicated gene is considered as a “poorly reproducible spot” and flagged.

Then “differentially expressed genes” were determined based on a cutoff value of the t-score. For both arrrays, genes were accepted as differentially expressed if |t-score|>3 for Cy3 and >4.0 for Cy5. If the t-score is a positive value, it means that the expression level in the Cy3 channel is higher than in the Cy5 channel. If it is a negative value, the expression level is in the reverse direction.

### Example 3

Microarray Analysis of A375 Melanoma Cells

One slide was analyzed with A375 melanoma cells treated with scFvMEL/rGel. Control cells labeled with cy5 and A375 ZR24 labeled with Cy3. The table shows the location of the spot on the array, average log intensity values (base 2), which show how good the signal was, the smoothed T scores which is used to determine the differentially expressed genes, the Cy5/Cy3 or Cy3/Cy5 ratio and the gene description. Negative smooth T values represent genes found to be inhibited. Positive smooth T values represent genes that are induced (Table II).

TABLE II
CG041359 Cy5 VRG24 control Km and Cy3 VRG24 Km (down-
regulated genes in VRG24 Km)

| | Average | Smoothed | | |
|---|---|---|---|---|
| Location1 | LogIntensity | TValue | Accession | Name |
| D10c10 | 11.1 | −6.0 | W68220 | KIAA0101 gene |
| | | | | product |
| C9d4 | 10.2 | −5.7 | X68303 | cyclin A2 |
| C6e6 | 10.3 | −5.6 | BC007101 | KIAA0101 gene |
| | | | | product |
| B8e8 | 10.5 | −4.6 | AA682613 | craniofacial devel- |
| | | | | opment protein 1 |
| C4d9 | 10.1 | −4.4 | NM_001786 | cell division cycle 2, |
| | | | | G1 to S and G2 |
| | | | | to M |
| C2c8 | 13.1 | −4.2 | AA423867 | multimerin |
| A12d6 | 11.1 | −4.2 | NM_004595 | spermine synthase |
| C2d1 | 9.9 | −4.1 | AA262211 | KIAA0008 gene |
| | | | | product |
| B7e6 | 11 .0 | −4.0 | NM_006452 | multifunctional |
| | | | | polypeptide similar |
| | | | | to SAICAR synthe- |
| | | | | tase and AIR |
| | | | | carboxylase |
| B1a7 | 11.5 | −4.0 | AL132777 | no match in |
| | | | | UniGene |
| C10c9 | 11.5 | −3.9 | AA460727 | adaptor-related pro- |
| | | | | tein complex 3, |
| | | | | sigma 1 subunit |
| B8e4 | 11.8 | −3.9 | NM_003051 | solute carrier family |
| | | | | 16 (monocarboxylic |
| | | | | acid transporters), |
| | | | | member 1 |
| B5e1 | 10.8 | −3.8 | AA452909 | no match in |
| | | | | UniGene |
| D7c1 | 10.5 | −3.8 | AA459292 | CDC28 protein |
| | | | | kinase 1 |
| C6b2 | 12.8 | −3.7 | AE003630 | no match in |
| | | | | UniGene |
| D11b7 | 11.7 | −3.7 | W33012 | transcription factor |
| | | | | Dp-1 |
| B11d1 | 10.3 | −3.6 | AA482324 | seladin-1 |
| B2b4 | 11.1 | −3.6 | NM_001233 | caveolin 2 |
| D7b9 | 10.6 | −3.5 | R28294 | glycine cleavage |
| | | | | system protein H |
| | | | | (aminomethyl |
| | | | | carrier) |
| C8c3 | 9.8 | −3.5 | AA504348 | topoisomerase |
| | | | | (DNA) II alpha |
| | | | | (170kD) |
| C9b7 | 11.0 | −3.5 | W81684 | transcription elon- |
| | | | | gation factor B |
| | | | | (SIII), polypeptide 1 |
| | | | | (15kD, elongin C) |
| C3e1 | 10.2 | −3.4 | AA156940 | programmed cell |
| | | | | death 5 |
| B4d7 | 10.8 | −3.4 | AA598676 | reticulocalbin 2, |
| | | | | EF-hand calcium |
| | | | | binding domain |
| B10a3 | 10.4 | −3.4 | N90630 | tyrosine 3-mono- |
| | | | | oxygenase/trypto- |
| | | | | phan 5-monooxy- |
| | | | | genase activation |
| | | | | protein, eta |
| | | | | polypeptide |
| D11d3 | 10.3 | −3.4 | AA129552 | forkhead box M1 |
| D8c7 | 12.4 | −3.3 | AA490721 | splicing factor, |
| | | | | arginine/serine- |
| | | | | rich 9 |
| C7c10 | 9.8 | −3.2 | D80000 | SMC1 (structural |
| | | | | maintenance of |
| | | | | chromosomes 1, |
| | | | | yeast)-like 1 |
| D6a9 | 11.1 | −3.2 | NM_006824 | nucleolar protein |
| | | | | p40; homolog of |
| | | | | yeast EBNA1- |
| | | | | binding protein |
| B3a7 | 12.2 | −3.2 | AA074666 | no match in |
| | | | | UniGene |
| C7c4 | 12.9 | −3.2 | NM_004856 | kinesin-like 5 |
| | | | | (mitotic kinesin- |
| | | | | like protein 1) |
| D2c8 | 11.1 | −3.2 | W87611 | nuclear factor I/B |
| B6c8 | 12.0 | −3.2 | T57556 | histidine triad |
| | | | | nucleotide-binding |
| | | | | protein |
| D1d9 | 10.8 | −3.1 | AA452909 | no match in |
| | | | | UniGene |
| B9e5 | 12.4 | −3.1 | AA676970 | phosphoglycerate |
| | | | | mutase 1 (brain) |
| A4b9 | 9.7 | −3.1 | AA025058 | RAB11A, member |
| | | | | RAS oncogene |
| | | | | family |
| B1d5 | 12.0 | −3.1 | AA251527 | karyopherin |
| | | | | (importin) beta 1 |
| C10e9 | 11.7 | −3.1 | H19203 | Putative: peroxire- |
| | | | | doxin 3 |
| D8a9 | 10.9 | −3.0 | H93087 | high-mobility group |
| | | | | (nonhistone chromo- |
| | | | | somal) protein 17 |
| B3c2 | 11 .3 | −3.0 | AA424833 | bone morphogenetic |
| | | | | protein 6 |

### Example 4

Microarray Analysis of Endothelial Cells

As described above, a slide was analyzed with human umbilical vascular endothelial cells (HUVEC) treated with VEGF/rGel . Control cells were labeled with cy5 and HUVEC ZR24 labeled with Cy3. The table shows the location of the spot on the array, average log intensity values (base 2), which show how good the signal was, the smoothed T scores which is used to determine the differentially expressed genes, the Cy5/Cy3 or Cy3/Cy5 ratio and the gene description. Negative smooth T values represent genes which were downregulated. Positive smooth T values represent genes which were upregulated (Table III).

TABLE III
G041359 Cy5 VRG24 control Km and Cy3 VRG24 Km (Upregulated
genes in VRG24 Km)

| | Average | Smoothed | | |
|---|---|---|---|---|
| Location1 | LogIntensity | TValue | Accession | Name |
| C7a4 | 7.7 | 13.0 | H39560 | Selectin E (endo- |
| | | | | thelial adhesion |
| | | | | molecule 1) |
| A11a6 | 8.4 | 10.6 | AA476272 | Tumor necrosis |
| | | | | factor, alpha- |
| | | | | induced protein 3 |
| B8d8 | 10.0 | 10.5 | NM_004856 | Kinesin-like 5 |
| | | | | (mitotic kinesin- |
| | | | | like protein 1) |
| D4e6 | 9.9 | 8.4 | R77293 | No match in |
| | | | | UniGene |
| A6a7 | 10.6 | 8.2 | W55872 | Nuclear factor of |
| | | | | kappa light poly- |
| | | | | peptide gene |
| | | | | enhancer in B-cells |
| | | | | inhibitor, alpha |
| C9a7 | 9.8 | 8.2 | T99236 | jun B proto- |
| | | | | oncogene |
| B4a7 | 10.4 | 7.9 | W55872 | Nuclear factor of |
| | | | | kappa light poly- |
| | | | | peptide gene |
| | | | | enhancer in B-cells |
| | | | | inhibitor, alpha |
| B10e4 | 7.9 | 7.7 | W69211 | Small inducible |
| | | | | cytokine subfamily |
| | | | | A (Cys—Cys), |
| | | | | member 11 |
| | | | | (eotaxin) |
| C3a4 | 7.2 | 6.6 | AA425102 | Small inducible |
| | | | | cytokine A2 |
| | | | | (monocyte |
| | | | | chemotactic protein |
| | | | | 1, homologous to |
| | | | | mouse Sigje) |
| D11c9 | 10.4 | 6.1 | AA127794 | KIAA0342 gene |
| | | | | product |
| D3e7 | 9.6 | 5.9 | XM_002964 | no match in |
| | | | | UniGene |
| B1e4 | 8.6 | 5.9 | W69211 | Small inducible |
| | | | | cytokine subfamily |
| | | | | A (Cys—Cys), |
| | | | | member 11 |
| | | | | (eotaxin) |
| D3a9 | 9.2 | 5.2 | AA479199 | Nidogen 2 |
| A7e5 | 9.5 | 4.7 | NM_000963 | Prostaglandin- |
| | | | | endoperoxide syn- |
| | | | | thase 2 (prostaglan- |
| | | | | din G/H synthase |
| | | | | and cyclooxy- |
| | | | | genase) |
| C2e5 | 10.9 | 4.6 | AA703141 | Erythrocyte mem- |
| | | | | brane protein band |
| | | | | 4.1 (elliptocytosis |
| | | | | 1, RH-linked) |
| C9c6 | 7.6 | 4.4 | AA040170 | Small inducible |
| | | | | cytokine A7 |
| | | | | (monocyte chemo- |
| | | | | tactic protein 3) |
| B11e4 | 8.0 | 4.3 | W69211 | Small inducible |
| | | | | cytokine subfamily |
| | | | | A (Cys—Cys), |
| | | | | member 11 |
| | | | | (eotaxin) |
| A12d2 | 9.1 | 4.3 | H57830 | H1 histone family, |
| | | | | member 0 |
| D5e9 | 9.1 | 4.3 | NM_005558 | Ladinin 1 |
| A1d3 | 10.1 | 4.2 | AA434130 | Thioredoxin reduc- |
| | | | | tase beta |
| B3c3 | 11.7 | 4.2 | AA284668 | Plasminogen activa- |
| | | | | tor, urokinase |
| A2a7 | 10.2 | 4.2 | AA031513 | Matrix metallopro- |
| | | | | teinase 7 |
| | | | | (matrilysin, uterine) |
| B10a9 | 9.2 | 4.2 | W65461 | Dual specificity |
| | | | | phosphatase 5 |
| B12e9 | 8.1 | 4.1 | AL034379 | no match in |
| | | | | UniGene |
| D11b10 | 7.5 | 4.1 | NM_001964 | Early growth |
| | | | | response 1 |
| D9c8 | 9.1 | 3.9 | NM_000720 | Calcium channel, |
| | | | | voltage-dependent, |
| | | | | L type, alpha 1D |
| | | | | subunit |
| D8d1 | 8.9 | 3.9 | AA453105 | H2A histone family, |
| | | | | member L |
| D9d4 | 10.1 | 3.7 | H87496 | TATA box binding |
| | | | | protein (TBP)-asso- |
| | | | | ciated factor, RNA |
| | | | | polymerase I, C, |
| | | | | 110kD |
| A9c8 | 11.1 | 3.7 | AA486919 | Triosephosphate |
| | | | | isomerase 1 |
| C12c7 | 9.2 | 3.6 | AA434487 | NGFI-A binding |
| | | | | protein 2 (ERG1 |
| | | | | binding protein 2) |
| D5e3 | 8.9 | 3.5 | N70734 | Troponin T2, |
| | | | | cardiac |
| C10c3 | 9.3 | 3.5 | AA453202 | Nuclear receptor |
| | | | | subfamily 1, group |
| | | | | D, member 1 |
| A10e5 | 8.5 | 3.5 | N30553 | Pregnancy specific |
| | | | | beta-1-glycoprotein |
| | | | | 4 |
| B8c8 | 9.8 | 3.5 | AC007229 | Dynamin 2 |
| B4a4 | 10.4 | 3.5 | AA011215 | Spermidine/ |
| | | | | spermine N1-acetyl- |
| | | | | transferase |
| C8e7 | 8.4 | 3.5 | N32768 | Pregnancy specific |
| | | | | beta-1-glycoprotein |
| | | | | 3 |
| A1d6 | 9.6 | 3.3 | AA446290 | Suppression of |
| | | | | tumorigenicity 5 |
| B1d2 | 9.4 | 3.3 | AA458533 | Forkhead box J1 |
| C12e3 | 9.1 | 3.3 | X52486 | Uracil-DNA |
| | | | | glycosylase 2 |
| B12d7 | 9.7 | 3.3 | AA135289 | Glutathione perox- |
| | | | | idase 2 (gastro- |
| | | | | intestinal) |
| A3a10 | 7.5 | 3.3 | T64134 | Small inducible |
| | | | | cytokine subfamily |
| | | | | A (Cys—Cys), |
| | | | | member 13 |
| D9c3 | 10.1 | 3.3 | AF026276 | Troponin T3, |
| | | | | skeletal, fast |
| B6b5 | 8.6 | 3.3 | H48706 | Baculoviral IAP |
| | | | | repeat-containing 3 |
| A12d9 | 8.7 | 3.2 | T71879 | Complement |
| | | | | component 2 |
| D7c5 | 11.9 | 3.1 | T62491 | Chemokine (C-X-C |
| | | | | motif), receptor 4 |
| | | | | (fusin) |
| B5d3 | 9.9 | 3.1 | AA489246 | Suppression of |
| | | | | tumorigenicity 14 |
| | | | | (colon carcinoma, |
| | | | | matriptase, epithin) |
| D9d3 | 9.5 | 3.1 | AJ341632 | no match in |
| | | | | UniGene |

### Example 5

Identification of Therapuetic Agents in HUVEC Cells Treated Immunotoxin

One example of a gene identified as being downregulated by immunotoxin therapy in HUVEC cells is topoisomerase II (Table II), which is involved in catalyzing the relaxation of supercoiled DNA by transient cleavage and religation of both strands of the DNA helix. By methods of the present invention inhibitors of topoisomerase II such as etoposide and anthracylcines such as doxorubicin, are identified as therapuetic agents that further promote the downregulation of topoisomerase gene expression and activity of cellular products thereof. By methods of the present invention, it is proposed that these therapeutic agents may be administered to a patient in combination with immunotoxin therapy to treat a disease such as a hyperproliferative disease by downregulating topoisomerase II gene expression and activity and cellular products thereof.

Another example of a gene identified as being downregulated by immunotoxin therapy in HUVEC cells (Table II), is spermine synthase. Spermine belongs to the group of polyamines which are essential for cell proliferation, differentiation and transformation, and is often found to be abundant in human tumors. Thus, in accordance with the methods of the present invention, inhibitors of spermine synthase such as the polyamine inhibitors N-(3-aminopropyl)cyclohexylamine (APCHA), N-cyclohexyl-1,3-diaminopropane (C-DAP), N-(n-butyl)-1,3-diaminopropane, S-adenosyl-1,12-diamino-3-thio-9-azadodecane (AdoDatad), difluoromethylomithine (DFMO), methyl glyoxal bis guanylhydrazone (MGBG), and methylglyoxal-bis(cyclopentylamidinohydrazone) MGBCP are identified as therapuetic agents to further promote the downregulation of spermine synthase expression and activity, and cellular products thereof. These therapeutic agents, in accordance with the present invention, may be administered to a patient in combination with immunotoxin therapy to treat a disease such as a hyperproliferative disease, by downregulating spermine synthase expression and activity.

### Example 6

Analysis of Microarray Data of Genes with High Levels of Induction

As described in Example 4 above, a slide was analyzed with human umbilical vascular endothelial cells (HUVEC) treated with VEGF/rGel. The table III indicates the genes that were differentially expressed. To confirm previous observations of genes upregulated, RT-PCR analysis was performed on the genes that showed the highest level of induction namely: E-Selectin (SELE), cytokine A2 (SCYA2), tumor necrosis factor alpha induced protein 3(TNFAIP3) and NFKB inhibitor alpha (NFKBIA).

Primers were designed based on the accession numbers from the microarray and confirmation of homology using Blast (NCBI). GAPDH primers were made as controls. The primers were as follows: SELE forward 5′GGTTTGGTGAGGTGTGCTC (SEQ ID NO: 1), SELE reverse 5′TGATCTTTCCCGGAACTGC (SEQ ID NO: 2), SCYA2 forward 5′TCTGTGCCTGCTGCTCATAG (SEQ ID NO: 3), SCYA2 reverse 5′TGGAATCCTGAACCCACTTC (SEQ ID NO: 4), TNFAIP3 forward 5′ATGCACCGATACACACTGGA (SEQ ID NO: 5), TNFAIP3 reverse 5′CGCCTTCCTCAGTACCAAGT (SEQ ID NO: 6), NFKBIA forward 5′AACCTGCAGCAGACTCCACT (SEQ ID NO: 7), NFKBIA reverse 5′GACACGTGTGGCCATTGTAG (SEQ ID NO: 8), GAPDH forward 5′GTCTTCACCACCATGGAG (SEQ ID NO: 9), GAPDH reverse 5′CCACCCTGTTGCTGTAGC SEQ ID NO: 10).

Human umbilical vein endothelial cells (HUVECs) were grown in 10 cm culture dishes and were either left untreated, or treated with an IC₅₀ dose of VEGF₁₂₁/rGel for 4 h and 24 h. As descsribed in Example 1, the cells were harvested and total RNA was isolated using TRIzol Reagent (Life Technologies, Inc., Gaithersburg, Md.). The integrity of RNA was verified by agarose gel electrophoresis and UV absorbance. First-strand cDNA was synthesized as described by the manufacturer of the Superscript First Strand Synthesis System (Invitrogen). RT-PCR was performed using a Minicycler PCR machine (MJ Research, Inc.).

All PCR products were of the size expected. Normalized for GAPDH, all four of the PCR products showed an increase upon treatment with VEGF₁₂₁/rGel, verifying the results seen in the original microarray. The results suggest that 1) the results obtained with microarray analysis are reliable and 2) treatment of HUVECs with VEGF₁₂₁/rGel results in the increase in the RNA levels of several genes that are involved in intermediary metabolic and inflammation pathways.

All of the compositions and methods disclosed and claimed herein can be made and executed without undue experimentation in light of the present disclosure. While the compositions and methods of this invention have been described in terms of embodiments, it will be apparent to those of skill in the art that variations may be applied to the compositions and methods, and in the steps or in the sequence of steps of the methods, described herein without departing from the concept, spirit, and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.

U.S. Pat. No. RE37,462

U.S. Pat. No. 5,359,046

U.S. Pat. No. 6,099,842

WO 89/00999

Atkinson et al., Biochem Molec. Biol., 276(30):27930-27935, 2001.

Bacterial Toxins and Cell Membranes, Jelajaszewicz and Wadstrom (Eds), Academic Press, 291, 1978.

Bakhshi et al., Cell, 41(3):899-906, 1985.

Bedzyk et al., J. Biol. Chem., 265:18615-20, 1990.

Bolognesi et al., Br. J. Haematol., 110(2):351-361, 2000.

Bonavida et al., Int J Oncol., 15:793-802, 1999.

Bonavida et al., Proc Natl. Acad. Sci. USA, 97:1754-9, 2000.

Chaudhary et al., Proc. Natl. Acad. Sci. USA, 87:9491-4, 1990.

Cleary and Sklar, Proc. Natl. Acad. Sci. USA, 82(21):7439-43, 1985.

Cleary etal., J. Exp. Med., 164(1):315-20, 1986.

Culver et al., Science, 256:1550-1552, 1992.

DeRisi et al, Nature Genetics, 14(4):457-460, 1996.

Falasca et al., Biochem J., 207(3):505-5059, 1982.

Gliniak et al., Cancer Res., 59:6153-8, 1999.

Gopal, Mol. Cell Biol., 5:1188-1190, 1985.

Graham and Van Der Eb, Virology, 52:456-467, 1973.

Kaneta et al, Jpn J. Cancer Res., 89(5):583-588, 1998.

Keane etal., Cancer Res., 59:734-41, 1999.

Mitchell et al., Ann. N.Y. Acad. Sci., 690:153-166, 1993.

Mitchell et al., J. Clin. Oncol., 8(5):856-859, 1990.

Morton and Ravindranath, In Tumor Immunology, Dalgleish (Ed.), London: Cambridge University Press, 1-55, 1996.

Morton et al., Ann. Surg., 216:463-482, 1992.

Pagliaro et al., Clin. Cancer Res., 4(8):1971-1976, 1998.

Ramakrishnan and Houston, Cancer Res., 44(4): 1398-404, 1984.

Ravindranath and Morton, Intern. Rev. Immunol., 7: 303-329, 1991.

Remington's Pharmaceutical Sciences, 15^{th} ed., 1035-1038 and 1570-1580, Mack Publishing Company, Easton, Pa., 1980.

Rosenberg et al., Ann. Surg., 210:474, 1989.

Rosenberg et al., N. Engl. J. Med., 319:1676, 1988.

Rosenblum et al., J Interferon Cytokine Res., 15(6):547-555, 1995.

Schena et al., Proc. Natl. Acad. Sci. USA, 93(20):10614-10619, 1996.

Schena et al., Science, 270(5235):467-470, 1995.

Sivan et al., Cancer Res., 47:3169-3173, 1987.

Stirpe et al., J. Biol. Chem., 255(14):6947-6953, 1980.

Thorpe et al., J. Natl. Cancer Inst, 75(1):151-159, 1985.

Tsujimoto and Croce, Proc. Natl. Acad. Sci. USA, 83(14):5214-8, 1986.

Tsujimoto et al., Science, 228(4706):1440-3, 1985.

Wagner et al., Science, 260:1510-1513, 1990.

Yeung et al., Int. J. Pept. Protein Res., 31(3):265-8, 1988.

What is claimed:

1. A method of identifying one or more genes or gene products that responds to immunotoxin therapy comprising administering an immunotoxin to a cell and determining one or more genes or gene products whose expression is upregulated or downregulated in response to the immunotoxin therapy.

2. The method of claim 1, wherein the cell is a cell in a diseased state.

3. The method of claim 2, wherein the cell is a hyperproliferative cell.

4. The method of claim 3, wherein the hyperproliferative cell is a cancer cell or an atherosclerosis cell.

5. The method of claim 4, wherein the cancer cell is a cell of the bladder, blood, bone, bone marrow, brain, breast, colon, esophagus, gastrointestine, gums, head & neck, kidney, liver, lung, nasopharynx, ovary, prostate, skin, stomach, testis, tongue, or uterus.

6. The method of claim 1, wherein the cell is located in a mammal.

7. The method of claim 6, wherein the mammal is a human.

8. The method of claim 1, wherein the cell is located in a cell culture.

9. The method of claim 1, wherein identifying the one or more genes or gene products comprises assessing the expression of one or more genes or gene products both before and after administration of the immunotoxin to the cell.

10. A method of claim 1, further characterized as comprising:
(a) administering the immunotoxin to a patient or a cell; and
(b) identifying one or more immunotoxin regulated genes or gene products that are upregulated or downregulated in response to the immunotoxin administration.

11. The method of claim 10, wherein the gene or gene product identified as being downregulated by immunotoxin therapy is topoisomerase II.

12. The method of claim 10, wherein the gene or gene product identified as being downregulated by immunotoxin therapy is spermine synthase.

13. The method of claim 10, wherein the gene or gene product identified as being downregulated is selected from the group consisting of the genes listed in Table II.

14. The method of claim 10, wherein the gene or gene product identified as being upregulated by immunotoxin therapy is E-selectin.

15. The method of claim 10, wherein the gene or gene product identified as being upregulated by immunotoxin therapy is cytokine A2.

16. The method of claim 10, wherein the gene or gene product identified as being upregulated by immunotoxin therapy is TNF-α induced protein 3.

17. The method of claim 10, wherein the gene or gene product identified as being upregulated by immunotoxin therapy is NFκB inhibitor alpha.

18. The method of claim 10, wherein the gene or gene product identified as being upregulated is selected from the group consisting of the genes listed in Table III.

19. The method of claim 10, wherein administration of immunotoxin therapy to a patient is by systemic intravenous injection, regional administration via blood or lymph supply, or directly to an affected site.

20. The method of claim 10, wherein the cell is a hyperproliferative cell.

21. The method of claim 20, wherein the hyperproliferative cell is a cancer cell or an atherosclerosis cell.

22. The method of claim 1, further comprising identifying a therapeutic agent or treatment regimen that will complement immunotoxin therapy comprising the steps of:
(a) identifying one or more regulated genes or gene products that are upregulated or downregulated in response to immunotoxin therapy in a patient undergoing said therapy;
(b) identifying one or more second agents or therapies that will promote a further upregulation or downregulation of one or more of the immunotoxin regulated genes.

23. The method of claim 22, further comprising administering the second agent or therapy to a patient.

24. The method of claim 22, wherein the gene or gene product identified as being downregulated is selected from the group consisting of the genes listed in Table II.

25. The method of claim 22, wherein the gene or gene product identified as being downregulated by immunotoxin therapy is topoisomerase II.

26. The method of claim 22, wherein the second agent is an inhibitor of topoisomerase II.

27. The method of claim 26, wherein the inhibitor of topoisomerase II is etoposide or doxorubicin.

28. The method of claim 26, wherein the inhibitor of topoisomerase II further promotes the downregulation of topoisomerase gene expression and activity, and cellular products thereof.

29. The method of claim 22, wherein the gene or gene product identified as being downregulated by immunotoxin therapy is spermine synthase.

30. The method of claim 22, wherein the second agent is an inhibitor of spermine synthase.

31. The method of claim 30, wherein the inhibitor of spermine synthase is a polyamine inhibitor.

32. The method of claim 31, wherein the polyamine inhibitor is N-(3-aminopropyl)cyclohexylamine (APCHA), N-cyclohexyl-1,3-diaminopropane (C-DAP), N-(n-butyl)-1,3-diaminopropane, S-adenosyl-1,12-diamino-3-thio-9-azadodecane (AdoDatad), difluoromethylomithine (DFMO), methyl glyoxal bis guanylhydrazone (MGBG), or methylglyoxal-bis(cyclopentylamidinohydrazone) MGBCP.

33. The method of claim 30, wherein the inhibitor of spermine synthase further promotes the downregulation of spermine synthase expression and activity, and cellular products thereof.

34. The method of claim 22, wherein the gene or gene product identified as being upregulated is selected from the group consisting of the genes listed in Table III.

35. The method of claim 22, wherein the gene or gene product identified as being upregulated by immunotoxin therapy is E-selectin.

36. The method of claim 22, wherein the second agent is an inducer of E-selectin.

37. The method of claim 36, wherein the inducer of E-selectin is TNF, lipopolysaccharide, lymphotoxin, or IL-1.

38. The method of claim 37, wherein the inducer of E-selectin further promotes the upregulation of E-selectin expression and activity, and cellular products thereof.

39. The method of claim 22, wherein the gene or gene product identified as being upregulated by immunotoxin therapy is cytokine A2.

40. The method of claim 22, wherein the second agent is an inducer of cytokine A2.

41. The method of claim 40, wherein the inducer of cytokine A2 is heme, lysophosphatidylcholine, interferon-gamma, IL-17, TNF, or IL-4.

42. The method of claim 41, wherein the inducer of cytokine A2 further promotes the upregulation of cytokine A2 expression and activity, and cellular products thereof.

43. The method of claim 22, wherein the gene or gene product identified as being upregulated by immunotoxin therapy is TNF-α induced protein 3.

44. The method of claim 22, wherein the second agent is an inducer of TNF-α induced protein 3.

45. The method of claim 44, wherein the inducer of TNF-α induced protein 3 (TNFAIP3) is TRAIL, Fas, CD40, PMA, UV, EBV, IL-1, or LPS.

46. The method of claim 45, wherein the inducer of TNF-α induced protein 3 further promotes the upregulation of TNF-α induced protein 3 expression and activity, and cellular products thereof.

47. The method of claim 22, wherein the gene or gene product identified as being upregulated by immunotoxin therapy is NFκB inhibitor alpha.

48. The method of claim 22, wherein the second agent is an inducer of NFκB inhibitor alpha.

49. The method of claim 48, wherein the inducer of NFκB inhibitor alpha is protein REIA, V-REL or deoxycholate(DOC).

50. The method of claim 49, wherein the inducer of NFκB inhibitor alpha further promotes the upregulation of NFκB inhibitor alpha expression and activity, and cellular products thereof.

51. The method of claim 22, wherein the therapeutic agent is an immunotoxin, fusion protein or immunoconjugate thereof.

52. The method of claim 22, wherein the therapeutic agent is a protein or a nucleic acid expression construct.

53. The method of claim 22, wherein the therapeutic agent is an antisense construct, or a small organic or inorganic molecule, or organo-pharmaceutical.

54. The method of claim 22, wherein the therapeutic agent is a DNA damaging agent, an alkylating agent, or an antitumor agent.

55. The method of claim 22, wherein the treatment regimen is a radiotherapy, immunotherapy, hormonal therapy or gene therapy.

56. A method of treating a patient with a hyperproliferative disease or condition comprising the steps of:
(a) administering to the patient an amount of an immunotoxin that is effective to treat a disease that is amenable to immunotoxin therapy; and
(b) administering to the patient an effective amount of a therapeutic agent or treatment regimen that is selected from the immunotoxin based changes in gene expression.

57. The method of claim 56, wherein the therapeutic agent or treatment regimen is selected through the practice of claim 1.

58. The method of claim 56, wherein the therapeutic agent is an inhibitor of topoisomerase II.

59. The method of claim 58, wherein the inhibitor of topoisomerase II is etoposide or doxorubicin.

60. The method of claim 56, wherein the therapeutic agent is a spermine synthase inhibitor.

61. The method of claim 60, wherein the inhibitor of spermine synthase is a polyamine inhibitor.

62. The method of claim 61, wherein the polyamine inhibitor is N-(3-aminopropyl)cyclohexylamine (APCHA), N-cyclohexyl-1,3-diaminopropane (C-DAP), N-(n-butyl)-1,3-diaminopropane, S-adenosyl-1,12-diamino-3-thio-9-azadodecane (AdoDatad), difluoromethylomithine (DFMO), methyl glyoxal bis guanylhydrazone (MGBG), or methylglyoxal-bis(cyclopentylamidinohydrazone) MGBCP.

63. The method of claim 56, wherein the therapeutic agent is an inducer of E-selectin.

64. The method of claim 63, wherein the inducer of E-selectin is lipopolysaccharide, lymphotoxin, or IL-1.

65. The method of claim 56, wherein the therapeutic agent is an inducer of cytokine A2.

66. The method of claim 65, wherein the inducer of cytokine A2 is heme, lysophosphatidylcholine, interferon-gamma, IL-17, TNF, or IL-4.

67. The method of claim 56, wherein the therapeutic agent is an inducer of TNF-α induced protein 3.

68. The method of claim 67, wherein the inducer of TNF-α induced protein 3 is TRAIL, Fas, CD40, PMA, UV, EBV, IL-1, or LPS.

69. The method of claim 56, wherein the therapeutic agent is an inducer of NFκB inhibitor alpha.

70. The method of claim 69, wherein the inducer of NFκB inhibitor alpha is REIA, V-REL or deoxycholate(DOC).

71. The method of claim 56, wherein the therapeutic agent may be administered to a patient in combination with immunotoxin therapy to treat a disease by downregulating spermine synthase expression and activity.

72. The method of claim 56, wherein the therapeutic agent may be administered to a patient in combination with immunotoxin therapy to treat a disease by downregulating topoisomerase II expression and activity.

73. The method of claim 56, wherein the therapeutic agent may be administered to a patient in combination with immunotoxin therapy to treat a disease by downregulating a gene selected from the group consisting of the genes listed in Table II.

74. The method of claim 56, wherein the therapeutic agent may be administered to a patient in combination with immunotoxin therapy to treat a disease by upregulating E-selectin expression and activity.

75. The method of claim 56, wherein the therapeutic agent may be administered to a patient in combination with immunotoxin therapy to treat a disease by upregulating cytokine A2 expression and activity.

76. The method of claim 56, wherein the therapeutic agent may be administered to a patient in combination with immunotoxin therapy to treat a disease by upregulating TNF-α induced protein 3 expression and activity.

77. The method of claim 56, wherein the therapeutic agent may be administered to a patient in combination with immunotoxin therapy to treat a disease by upregulating NFκB inhibitor alpha expression and activity.

78. The method of claim 56, wherein the therapeutic agent may be administered to a patient in combination with immunotoxin therapy to treat a disease by upregulating a gene selected from the group consisting of the genes listed in Table III.

79. The method of claim 56, wherein the therapeutic agent is an immunotoxin, fusion protein or immunoconjugate thereof.

80. The method of claim 56, wherein the therapeutic agent is a protein or a nucleic acid expression construct.

81. The method of claim 56, wherein the therapeutic agent is an antisense construct, or a small organic or inorganic molecule, or organo-pharmaceutical.

82. The method of claim 56, wherein the therapeutic agent is a DNA damaging agent, an alkylating agent, or an antitumor agent.

83. The method of claim 56, wherein the treatment regimen is a radiotherapy, immunotherapy, hormonal therapy or gene therapy.

84. The method of claim 56, wherein administration of immunotoxin therapy and/or a therapeutic agent is by systemic intravenous injection, regional administration via blood or lymph supply, or directly to an affected site.

85. The method of claim 56, wherein the hyperproliferative disease is a cancer.

86. The method of claim 85, wherein the cancer is a cancer of the bladder, blood, bone, bone marrow, brain, breast, colon, esophagus, gastrointestine, gums, head, kidney, liver, lung, nasopharynx, neck, ovary, prostate, skin, stomach, testis, tongue, or uterus.

87. The method of claim 56, wherein the hyperproliferative disease or condition is atherosclerosis.
